(19) United States
(12) Patent Application Publication
(10) Pub. No.: US 2021/0165936 A1
(43) Pub. Date: Jun. 3, 2021

(54) PARALLEL SEQUENTIAL DERIVATIVE COMPUTATION FOR PROCESS SIMULATION

(71) Applicant: HONEYWELL INTERNATIONAL INC., Morris Plains, NJ (US)

(72) Inventors: David Pavan, Calgary (CA); Jeffrey Renfro, Deer Park, TX (US); Jianye Zhu, Calgary (CA); Christopher Daniel Elliott, Calgary (CA); Mitchell Roy Craig, Calgary (CA)

(21) Appl. No.: 16/701,298

(22) Filed: Dec. 3, 2019

(51) Int. Cl. G06F 30/27

(52) U.S. Cl. CPC G06F 30/27; G06F 2111/10

(57) ABSTRACT

A simulation system includes independent processing units each having a processor, memory, and a mathematical solver. An additional independent processing unit has an additional memory storing a predictive simulation model that includes equations relating manipulated variables, orchestration logic, and a master optimization solver. The orchestration logic initiates the additional independent processing unit distributing to each independent processing unit concurrent timing information, instructions to each perturb one manipulated variable, and values for the manipulated variables. The independent processing units concurrently execute the predictive model to perturb their manipulated variable to calculate derivatives for each manipulated variable. The additional independent processing unit receives the derivatives, calculates new variable values for each manipulated variable using the derivatives, and then runs the predictive model with the new variable values. The master optimization solver evaluates simulation results obtained from the predictive model using the new variable values relative to an objective.

### FIELD

Disclosed embodiments relate to process simulation.

### BACKGROUND

Process simulation software is based on modeling a real phenomenon with a set of mathematical formulas. Simulation generally comprises a computer program that allows the user to observe an operation through simulation without actually performing that operation. Simulation software is used widely to design equipment so that the final product will be as close to design specifications as possible without expensive process modification. Advanced computer programs can simulate power systems, weather conditions, electronic circuits, chemical reactions, mechatronics, heat pumps, feedback control systems, atomic reactions, and complicated biological processes. In theory, any real phenomena that can be reduced to mathematical data and equations can be simulated on a computer.

Commercially available chemical process simulation software generally follows one of two technical approaches. These approaches are sequential modular where individual simulation models are converged to solution in connected sequence, or simultaneous where all simulation models are solved together as a system of equations and unknowns. The sequential modular approach is the most commonly used approach due to its advantages of robust initialization and reliable convergence of individual, autonomous models connected by material and heat flows.

Economic optimization of extant chemical process facilities is commercially and environmentally valuable. Economic optimization requires an objective to be maximized or minimized such as cost, yield, or profit, a predictive simulation model that describes the behavior and constraints of the process facility, and a set of manipulated variables of the predictive model to be manipulated to approach the objective within the simulation model constraints.

Optimization solvers typically employ successive quadratic programming (SQP) or Primal-Dual (‘Newton-like’) methods which require derivative information comprising the derivatives of the respective manipulated variables. Optimization solvers seek to minimize or maximize quantities and require the derivative information for the manipulated variables to determine how to change their values to converge on a maximum or minimum. Sequential process models do not typically provide derivative information because their equations are internally combined with local solver methods which prohibit explicit differentiation, as opposed to isolated sets of equations as in a simultaneous equation-based simulator. It is therefore necessary to perturb these manipulated variables and converge the model on each perturbation to calculate numerical derivatives by finite difference methods.

### SUMMARY

This Summary is provided to introduce a brief selection of disclosed concepts in a simplified form that are further described below in the Detailed Description including the drawings provided. This Summary is not intended to limit the claimed subject matter's scope.

Disclosed embodiments recognize known manipulated variable perturbing predictive process simulation models for determining derivatives for each simulation model manipulated variable is known to be time-consuming, and the time required grows linearly with the number of model variables and the number of iterations to optimize the process for a complex processing facility. Generally, model simulations for commercially valuable chemical processes, such as a refining or distillation process, are especially time-consuming to solve because sequential models using conventional serial sequential approaches may require hours or even days of computation time to converge to a solution.

For continuously running processes with a variable demand and variable feedstock and other input variables such as cost-variable power, the time to optimize (meaning to converge to an optimal solution) the process may prove prohibitive for useful economic optimization. The conventional approach for reducing the optimization time is to replace the sequential modular predictive model with an equation-based simultaneous model that provides analytical derivatives to remove the need to perturb for obtaining numerical derivative values. While this approach can generally significantly reduce the time required to converge the optimization process, this Disclosure recognizes that this approach requires simultaneous simulation models that are far less common than sequential simulation models and often more challenging to solve due to highly nonlinear equation forms and the need to generate high quality initial estimates to aid in convergence.

Disclosed aspects include parallel sequential process simulation systems and methods. A method of process simulation comprises providing a plurality of independent processing units each having a memory associated with a processor and an optimization or non-optimization solver. An additional independent processing unit has an additional processor and an additional memory storing a predictive simulation model that includes a set of equations which relate a plurality of manipulated variables, orchestration logic, and an optimization solver.

The orchestration logic is for initiating the additional independent processing unit communicating with the independent processing units including distributing information to each of the plurality of independent processing units comprising concurrent timing information, and instructions to enable each independent processing unit to perturb a different manipulated variable, and values for the respective manipulated variables. The values for the manipulated variables are initial values for the manipulated variables in the first iteration of the method and updated variable values in the case of subsequent iterations of the method. Although the simulation model needs to be consistent across all processing units in the simulation system, the source of the single simulation model used throughout the simulation system can be from the additional independent processing unit, or alternatively the simulation model can be received from a central repository that all the independent processing units, including additional independent processing unit, acquires the simulation model from.

The independent processing units each using their mathematical solver concurrently execute the predictive simulation model to each perturb a different one of the manipulated variables to calculate a derivative for their respective manipulated variables by comparing to previous values of their manipulated variable to determine derivative information, i.e. the direction of change in the manipulated variable which is needed by the master optimization solver to drive the solution toward the desired objective. The additional independent processing unit receives the derivatives calculated by each of the independent processing units.

The additional independent processing unit calculates new variable values for each of the plurality of manipulated variables using the derivatives, and then runs the predictive simulation model with the new variable values. The simulation results from the predictive simulation model run with the new variable values are evaluated relative to an objective. Given a predetermined tolerance from the objective it can be determined whether another iteration of the method is needed or not. During iterations the additional independent processing unit sends only the updated variable values to the respective independent processing units.

### BRIEF DESCRIPTION OF THE DRAWINGS

FIG. 1 depicts a simulation system including a single processing unit implementing steps for a known serial sequential method of process simulation.

FIG. 2 depicts a simulation system including a plurality of independent processing units and additional independent processing unit executing orchestration logic for implementing steps for a disclosed parallel sequential method of process simulation, according to an example embodiment.

### DETAILED DESCRIPTION

Disclosed embodiments are described with reference to the attached figures, wherein like reference numerals are used throughout the figures to designate similar or equivalent elements. The figures are not drawn to scale and they are provided merely to illustrate certain disclosed aspects. Several disclosed aspects are described below with reference to example applications for illustration. It should be understood that numerous specific details, relationships, and methods are set forth to provide a full understanding of the disclosed embodiments.

Disclosed aspects recognize when performing a mathematical optimization of a predictive simulation model using Newton-like methods it is necessary to provide the optimization solver with derivative information regarding the variables it manipulates. This is necessary to determine the direction of change in each of the model manipulated variables used to approach the objective.

It is recognized that a sequential modular predictive process simulation model cannot provide analytical derivatives for the manipulated variables because the model behavior is procedural and not strictly equation-based. It is therefore generally necessary to perturb each manipulated variable to calculate a numerical derivative at each iteration of the optimization. Perturbation applies a relatively small change to a manipulated variable value and then converges the simulation model. The change in output values is used to calculate a derivative for that manipulated variable at that point in the optimization. The optimization solver must perturb all manipulated variables independently to see the effect of each manipulated variable on the model. The optimization solver can then apply changes based on the derivatives to all manipulated variables of the model in aggregate to best approach the objective. This process is then repeated until the optimization solver is satisfied that it has achieved the objective given a reasonable tolerance relative to the objective.

Each model iteration for conventional process simulation model therefore requires numerous solutions of the predictive model, one for each manipulated variable to calculate the derivative information followed by at least one to apply new values using the derivatives to all manipulated variables. Solving the conventional process simulation model at each iteration is dependent on the completion of all perturbations, but each perturbation is independent of one another.

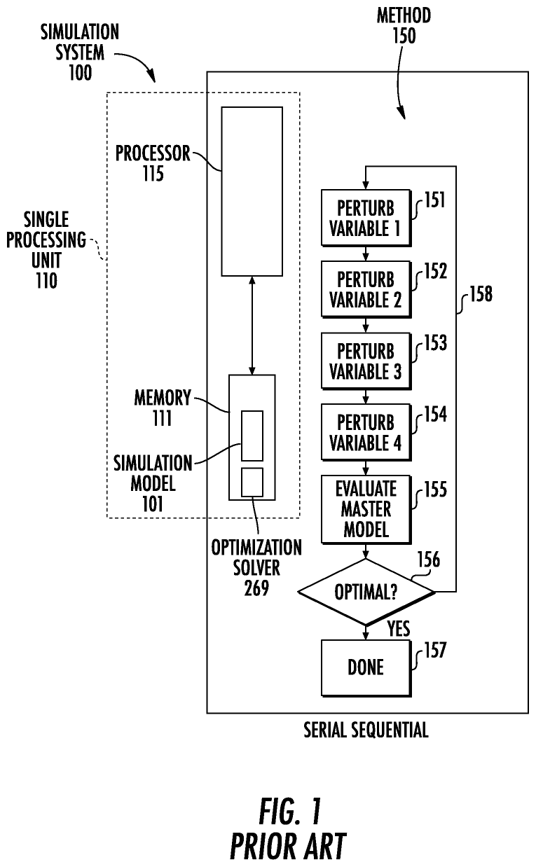

Disclosed aspects include a new process simulation technique that uses simultaneously running (concurrent or parallel) derivative calculations, where disclosed aspects distribute the perturbation of individual manipulated variables to obtain the respective derivatives using individual copies of the same predictive model executing on independent computing resources referred to herein as “processing units”. FIG. 1 depicts a conventional simulation system 100 comprising a single processing unit 110 implementing a conventional method 150 of serial sequential process simulation that for simplicity is shown having four manipulated variables shown as variable 1, variable 2, variable 3, and variable 4. As noted above, although described herein as being generally for a chemical process, disclosed aspects also apply to other processes and networks based on a real phenomenon, including electrical networks and mechanical networks.

The single processing unit 110 has only a single processor 115 with an associated memory 111 that stores a predictive simulation model 101 and an optimization solver 269, where the single processor 115 reads program instructions of the predictive simulation model 101 from the memory 111 and executes the program instructions to implement the simulation method 150. The program instructions are generally ordinary central processing unit (CPU) instructions, such as add, subtract, multiply, move data, branch, divide, and compare. The single processing unit 110 can run only one set of instructions at the same time, which for simulations is known as a serial sequential simulation which involves a plurality of different manipulated variables, which for method 150 involves perturbing the respective manipulated variables in a serial fashion, thus one manipulated variable being perturbed at any given time.

In step 151, the single processing unit 110 using the predictive simulation model 101 implements perturbing manipulated variable 1 to return an updated value for variable 1 enabling the simulation model 101 to calculate a new value for variable 1, where the difference between the new value of variable 1 and the original (or previous) value of variable 1 provides the derivative for variable 1. Upon completion of step 151, the method 150 then moves step 152 where the single processing unit 110 using the predictive simulation model 101 implements perturbing variable 2 to return an updated value for variable 2 enabling the calculation of a new value for variable 2, where the difference between the new value of variable 2 and the original (or previous) value of variable 2 provides the derivative for variable 2.

Upon completion of step 152, the method 150 then moves to step 153 where the single processing unit 110 using the predictive simulation model 101 implements perturbing variable 3 to return an updated value for variable 3 enabling the calculation of a new value for variable 3, where the difference between the new value of variable 3 and the original (or previous) value of variable 3 provides the derivative for variable 3. Upon completion of step 153, the method 150 then moves step 154 where the single processing unit 110 using the simulation model 101 implements perturbing variable 4 to return an updated value for variable 4 enabling the calculation of a new value for variable 4, where the difference between the new value of variable 4 and the original (or previous) value of variable 4 provides the derivative for variable 4.

Step 155 comprises evaluating the predictive simulation model 101 using the optimization solver stored in the memory 111 based on the derivative information received for the respective manipulated variables 1 to 4 generated in steps 151 to 154 to calculate new manipulated variable values for variables 1 to 4. Step 156 shown as “optimal” comprises determining whether the predictive simulation model 101 using the new variable values for variable 1 to 4 converges by determining whether a minimum or maximum for a predetermined simulation objective is reached. The optimization continues iterating until a minimum or maximum, depending on the goal, is met within a tolerance value, or alternatively until a maximum number of allowed iterations is reached to avoid running the optimization indefinitely.

An example objective to be reached is to maximize the purity of a product at a chemical processing plant at the cost of increasing some other quantity such as power consumption in the plant. If the objective considered in step 156 is found to be satisfied, the method proceeds to step 157 comprising completion of the serial sequential simulation shown in FIG. 1 as being “done”. If the objective considered in step 156 is found to not be satisfied, the method utilizes iteration step 158 which returns the method to step 151 to again perform the above described sequence of serial steps 151-155 using the new values for the manipulated variables until step 156 is satisfied that the process simulation has reached a minimum or maximum for the simulation objective, or as noted above alternatively until a maximum number of allowed iterations is reached to avoid running the optimization indefinitely.

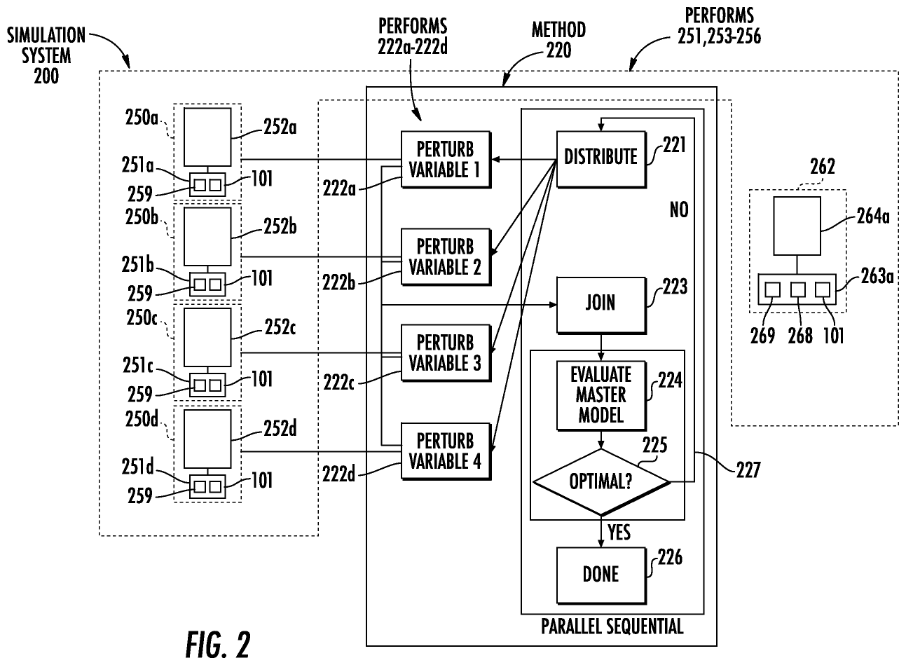

FIG. 2 depicts a disclosed simulation system 200 including a plurality of independent (parallel connected) processing units 250a, 250b, 250c and 250d, and an additional independent processing unit 262 executing orchestration logic 268, for implementing a disclosed method 220 of parallel sequential simulation, which for simplicity is again shown having four simulated measured variables shown as variable 1, variable 2, variable 3, and variable 4, according to an example embodiment. Each independent processing unit 250a-250d has an independent processor 252a-252d with an associated memory 251a-251d that stores a copy of the same predictive simulation model 101 received from the additional independent processing unit 262 or from another source for the simulation model 101, and a mathematical solver 259 comprising an optimization solver or a non-optimization solver.

The independent processing units 250a, 250b, 250c and 250d each read the same program instructions of the predictive simulation model 101 from their respective memory 251a-251d, and execute the program instructions to implement steps 252a to 252d for the parallel sequential simulation method 220 that involves perturbing variable 1, 2, 3 and 4, respectively. The mathematical solver 259 in the case it is an optimization solver can be the same solver as the master optimization solver 269. The predictive simulation model 101 that includes a set of equations which relate a plurality of manipulated variables.

The mathematical solvers 259 can converge its copy of the predictive simulation model 101 for manipulated variable changes (perturbation to determine derivative information) as directed by the orchestration logic 268 stored in the memory 263a of the additional processing unit 262 that also includes a master optimization solver 269 for solving the predictive simulation model 101. The additional independent processing unit 262 comprises an additional processor 264a. The master optimization solver 269 as noted above can be the same mathematical solver as the mathematical solver 259 associated with the independent processing unit 250a-250d in the case the mathematical solver 259 comprises an optimization solver. As noted above, the mathematical solver for each independent processing unit 250a to 250d can be either an optimizing or a non-optimizing solver. However, the master optimization process generally needs an optimizing solver 269.

Step 221 comprises the orchestration logic 268 initiating the additional processing unit 262 distributing to the plurality of independent processing units 250a-250d concurrent timing information, instructions to each perturb different ones of the plurality of manipulated variables, values for the plurality of manipulated variables, and optionally a copy of the predictive simulation model 101. As noted above, the plurality of independent processing units 250a-250d can also receive the predictive simulation model 101 from a source other than the additional independent processing unit, such as a central repository. In steps 222a to 222d the independent processing units 250a-250d each perturb for the specific manipulated variable derivative indicated by the orchestration logic 268 as directed by the master optimization solver 269 to concurrently each execute the predictive simulation model 101 to each perturb one of the plurality of manipulated variables, to collectively calculate derivatives for each of the manipulated variables.

Step 263 comprises the additional independent processing unit 262 receiving the derivatives from each of the plurality of independent processing units 250a-250d. Step 224 comprises the additional independent processing unit 262 calculating new variable values for each of the manipulated variables using the derivative information, and then running the predictive simulation model 101 with the new variable values. Step 225 comprises evaluating simulation results obtained from the predictive simulation model 101 generated by the master optimization solver 269 using the new variable values relative to an objective.

If the objective considered in step 225 is found to be satisfied, the method proceeds to step 226 comprising completion of the parallel sequential simulation shown in FIG. 2 as being “done”. If the objective considered in step 225 is found to not be satisfied, the method utilizes iteration step 227 which returns the method 220 to step 221 to again perform the above described sequence of serial steps 221-224 using the new values for the manipulated variables until step 225 is satisfied that the parallel sequential simulation has reached a minimum or maximum for the simulation objective, or as noted above alternatively until a maximum number of allowed iterations is reached to avoid running the optimization indefinitely.

The use of a plurality of independent processing units 250a, 250b, 250c and 250d for simultaneously implementing manipulated variable perturbing steps for a disclosed parallel sequential method of process simulation is unique and helpful because compared to conventional serial sequential method 150 described relative to FIG. 1, method 220 extracts a key aspect of the optimization calculation made to execute in parallel, that being the calculation of derivatives for the respective manipulated variables. Because conventional manipulated variable perturbation implemented by the single processor 115 of the single processing unit 110 shown in FIG. 1 described above occurs in series (calculation of one derivative for a manipulated variable followed by calculation of another derivative for another manipulated variable, and so on) as shown in FIG. 1, it occupies a significant and typically dominant portion of the total runtime for the optimization.

By disclosed aspects distributing the perturbations of the manipulated variables to multiple independent computing resources shown as processing units 250-250d, shown optionally with one processing unit for each manipulated variable to be perturbed, this significant runtime can be reduced linearly. For example, by performing two manipulated variable perturbations at once halves the required time; four concurrent manipulated variable perturbations quarter the runtime and so on to a maximum of the inverse of the total number of manipulated variables. Accordingly, the technical benefits from using disclosed parallel sequential are significant improvements in computational efficiency.

By using multiple independent computing resources for implementing a parallel sequential method of process simulation, process optimization with sequential modular predictive models becomes far more practical, especially for complex models with long convergence times and/or large numbers of manipulated variables. Given that the majority of existing process simulation tools and models are sequential modular this enables the benefits of process optimization for a larger pool of facilities and customers, especially those who cannot afford sophisticated equation-based modeling tools and expertise.

It is noted that the availability and affordability of on-premise multi-core computers can be an enabling feature for disclosed aspects. Each core is considered an independent computer for this Disclosure. Multi-core computing is functionally equivalent for the purposes of this Disclosure to a network or cluster of connected computing units. As known in the computer arts, a multi-core processor is a computer processor integrated circuit (IC) with two or more separate processing units, commonly called cores, each of which reads and executes program instructions, as if the computer had several processors. The instructions are ordinary CPU instructions, where the single processor can run instructions on separate cores at the same time, increasing the overall speed for programs that support multithreading or other parallel computing techniques. Some semiconductor manufacturers typically integrate the cores onto a single IC die (known as a chip multiprocessor) or onto multiple dies stacked vertically or place lateral to one another in a single package. The microprocessors currently used in most personal computers are currently multi-core. A multi-core processor implements multiprocessing in a single physical package.

One or more of the independent computing resources 250a-250d for a disclosed simulation system 200 can utilize cloud-based computer clusters, which is a relatively modern development enabling more affordable customer access to the benefits of this Disclosure. Disclosed aspects can solve the simulation customer problem whose business is selling and deploying optimization applications that is experiencing long solution times for their optimization problems, which was making their offering less competitive in the marketplace and application development was consuming an excessive amount of engineering man hours waiting for optimization solutions to complete.

Disclosed embodiments can be applied to generally to any process simulation that simulates a real phenomena. Optimization to minimize or maximize economic quantities of generally any mathematical simulation or a network involving a physical quantity is within the scope of this Disclosure. The predictive simulation models may be mathematical representations of generally any domain involving a real phenomena, including in the case of networks, to chemical, electrical, and mechanical networks.

While various disclosed embodiments have been described above, it should be understood that they have been presented by way of example only, and not limitation. Numerous changes to the subject matter disclosed herein can be made in accordance with this Disclosure without departing from the spirit or scope of this Disclosure. In addition, while a particular feature may have been disclosed with respect to only one of several implementations, such feature may be combined with one or more other features of the other implementations as may be desired and advantageous for any given or particular application.

1. A method of process simulation, comprising:
providing a plurality of independent processing units each having a processor, a memory and a mathematical solver, and an additional independent processing unit having an additional processor and an additional memory storing a predictive simulation model that includes a set of equations which relate a plurality of manipulated variables, orchestration logic and a master optimization solver,
the orchestration logic initiating the additional independent processing unit distributing information to the plurality of independent processing units including concurrent timing information, instructions to each perturb one of the plurality of manipulated variables, and values for the ones of the plurality of manipulated variables;
the independent processing units each concurrently executing the predictive simulation model to each perturb their one of the plurality of manipulated variables to collectively calculate derivatives for each of the plurality of manipulated variables;
the additional independent processing unit receiving the derivative from each of the plurality of independent processing units, wherein the additional independent processing unit calculates new variable values for each of the plurality of manipulated variables using the derivatives and then running the predictive simulation model with the new variable values, and
evaluating simulation results obtained from the predictive simulation model by the master optimization solver using the new variable values relative to an objective.

2. The method of claim 1, when it is determined that the objective has not been met further comprising distributing the new variable values to the plurality of independent processing units, and then repeating the concurrently executing, the receiving, the running, and the evaluating.

3. The method of claim 1, wherein at least one of the plurality of independent processing units and the additional processing unit are cloud-located.

4. The method of claim 1, wherein a total number of the plurality of independent processing units equals a number of the plurality of manipulated variables.

5. The method of claim 1, wherein a total number of the plurality of independent processing units is less than a number of the plurality of manipulated variables.

6. The method of claim 1, wherein the predictive simulation model is a chemical process simulation model configured for simulating a distilling process or a refining process.

7. The method of claim 1, wherein the predictive simulation model is for a mechanical network or an electrical network.

8. The method of claim 1, further comprising the additional independent processing unit distributing a copy of the predictive simulation model to the plurality of independent processing units.

9. The method of claim 1, wherein the plurality of independent processing units receive the predictive simulation model in the providing step from a source other than the additional independent processing unit.

10. A simulation system, comprising:
a plurality of independent processing units each having a processor, a memory, and a mathematical solver;
an additional independent processing unit having an additional processor and an additional memory storing a predictive simulation model that includes a set of equations which relate a plurality of manipulated variables, orchestration logic, and a master optimization solver;
the orchestration logic for initiating the additional independent processing unit distributing information including concurrent timing information, instructions to each perturb one of the plurality of manipulated variables, and values for the ones of the plurality of manipulated variables;
the independent processing units for each concurrently executing the predictive simulation model to each perturb their one of the plurality of manipulated variables to collectively calculate derivatives for each of the plurality of manipulated variables;
the additional independent processing unit coupled for receiving the derivative from each of the plurality of independent processing units, wherein the additional independent processing unit is for calculating new variable values for each of the plurality of manipulated variables using the derivatives and then running the predictive simulation model with the new variable values; and
the master optimization solver for evaluating simulation results obtained from the predictive simulation model using the new variable values relative to an objective.

11. The system of claim 10, wherein at least one of the plurality of independent processing units and the additional processing unit are cloud-located.

12. The system of claim 10, wherein a total number of the plurality of independent processing units equals a number of the plurality of manipulated variables.

13. The system of claim 10, wherein a total number of the plurality of independent processing units is less than a number of the plurality of manipulated variables.

14. The system of claim 10, wherein the predictive simulation model is a chemical simulation model configured for simulating a distilling process or for simulating a refining process.

15. The system of claim 10, wherein the predictive simulation model is configured for a mechanical network or an electrical network.

16. The system of claim 10, wherein the mathematical solver comprises an optimization solver.

17. The system of claim 10, further comprising the additional independent processing unit for distributing a copy of the predictive simulation model to the plurality of independent processing units.
